(19) United States
(12) Patent Application Publication
(10) Pub. No.: US 2010/0010089 A1
(43) Pub. Date: Jan. 14, 2010

(54) SYSTEM AND METHOD FOR PRODUCTION AND USE OF FULVIC ACID

(76) Inventors: Don Calvin Van Dyke, Orem, UT (US); Asa Staples Nielson, Orem, UT (US); Bruce Sutton, Provo, UT (US); Dan Davies, Fillmore, UT (US)

Correspondence Address: BRYAN C. BRYNER, SUITE 600, 215 SOUTH STATE ST., SALT LAKE CITY, UT, 84111, US

(21) Appl. No.: 12/500,501

(22) Filed: Jul. 9, 2009

Related U.S. Application Data
(60) Provisional application No. 61/079,312, filed on Jul. 9, 2008.

(51) Int. Cl. A01N 37/10; C05F 11/02; C07C 65/05

(52) U.S. Cl. 514/568; 71/24; 562/476

(57) ABSTRACT

A method and system for producing fulvic acid and humic acid comprises extracting a liquid from an organic compost mixture. The liquid may be extracted by collecting liquid that percolates from the organic compost mixture or by separating liquid from solid components in the organic compost mixture. The extracted product comprises fulvic acid in an amount of at least 4% by weight, and more specifically at least 7%, and humic acid in an amount less than approximately 3% by weight.

### RELATED APPLICATIONS

This application claims the benefit of U.S. Provisional Application No. 61/079,312, filed Jul. 9, 2008, which application is incorporated herein by reference in its entirety.

### BACKGROUND

Fulvic acid is a naturally-occurring organic product derived from humus, the organic material in soils produced by the decomposition of organic matter. In addition to fulvic acid, humus also contains humic acid and humin. These humic substances are active components in soil and provide numerous benefits for plants. Fulvic acid is the most plant-active of the humic substances. It is a plant growth stimulator that increases plant metabolism, nutrient intake, and improves root development.

Humic substances, including fulvic acid and humic acid, are largely found in pre-historic deposits of lignite, a soft, brownish coal that has developed from peat through bacterial action over millions of years. Smaller quantities are also found naturally in soil. Thus, while humic substances are naturally-occurring, extracting them from natural sources has proved to be complex and problematic. This is particularly true for extraction of fulvic acid from natural sources. For example, most traditional methods of extraction of fulvic acid in commercial quantities generally require extraction from lignite or coal, as is described in U.S. Pat. Nos. 4,788,360; 5,004,831; 5,248,814; 5,670,345; 5,854,032; and 6,695,892. Other known techniques involve extraction of humic substances from humic acid bearing mineral ores, such as U.S. Pat. No. 5,688,999. These methods generally require the use of acids and bases to leech out the desired components, and often involve many complex and energy intensive processes.

### SUMMARY

The inventors have observed that the known methods and systems for producing fulvic acid are complicated, expensive, inefficient, and harmful to the environment. It is thus desirable that fulvic acid and humic acid preparations be produced in a cheaper, faster, and easier process that is less harmful to the environment and from a more reliable source. The inventors have discovered that fulvic acid and humic acid compositions can be produced in this manner by extracting them from a source other than lignite and hardrock minerals.

In accordance with the novel system and method described herein fulvic acid and humic acid are extracted from an organic compost mixture by extracting the liquid component of the organic compost mixture. The liquid component may be extracted by collecting liquid percolating from the organic compost mixture, or by separating the liquid component from the solid components of the organic compost mixture. A liquid such as water that dissolves fulvic acid may be added to the organic compost mixture prior to extraction of the liquid component. The liquid component in the organic compost mixture may be separated from the solid components by means of a separator, such as a centrifuge, belt press, filter press, or membrane press. The novel method and system may include optional additional steps, including filtration and treatment of the organic compost mixture and/or effluent, and may reuse the liquid effluent or solids byproduct to optimize the quantity of fulvic acid and humic acid extracted. The novel system and method is thus able to produce fulvic acid and humic acid without the need or use of acids.

The resulting novel product produced by the novel method and system described herein contains fulvic acid in an amount of at least 4% by weight, and humic acid in an amount of up to about 3% by weight. The product more specifically comprises fulvic acid in an amount of approximately 4% to approximately 10%, and more specifically at least approximately 7% by weight, and more specifically approximately 7% to about 10% by weight. Because it is produced from an organic compost mixture, the product also contains micronutrients and macronutrients needed by plants, and contains few heavy minerals.

Because of its composition, the novel product can be used to improve the health of plants. In one aspect, the novel product can be used to eradicate pests from plants, including the bark beetle from coniferous trees.

### BRIEF DESCRIPTION OF THE DRAWINGS

The novel system, methods, and products described herein can be understood in light of FIGS. 1-4, in which:

FIG. 1 depicts an embodiment of one aspect of the novel system and method.

FIG. 2 depicts an embodiment of one aspect of the novel system and method.

FIG. 3 depicts an embodiment of one aspect of the novel system and method.

FIG. 4 depicts an embodiment of one aspect of the novel system and method.

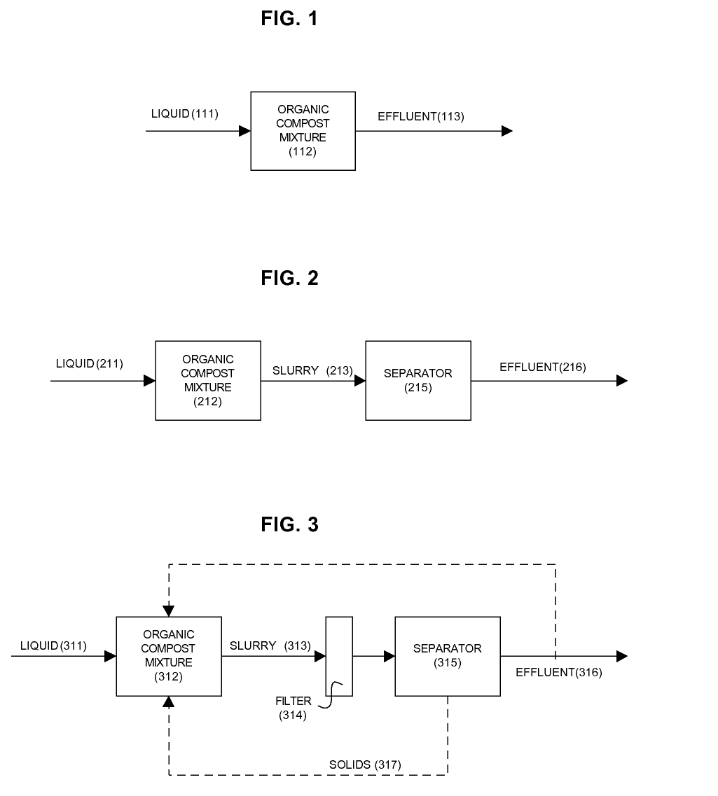

FIGS. 1-4 illustrate specific aspects of the novel system, methods, and products described herein and constitute a part of the specification. Together with the following description, the Figures demonstrate and explain the principles of the products and processes.

### DETAILED DESCRIPTION

The following description includes specific details in order to provide a thorough understanding of the novel method and system of producing fulvic acid. The skilled artisan will understand, however, that the products and methods described below can be practiced without employing these specific details, or that they can be used for purposes other than those described herein. Indeed, they can be modified and can be used in conjunction with products and techniques known to those of skill in the art in light of the present disclosure.

Reference in the specification to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment

One embodiment of the novel system and method for producing humic substances, including fulvic acid, is shown in FIG. 1. In this system and method, a source liquid (111) is first combined with an organic compost mixture (112). After the liquid (111) has been combined with the organic compost mixture (112), the liquid component of the organic compost mixture is then extracted from the organic compost mixture (112). The resulting liquid effluent (113) contains, among other components, fulvic acid and humic acid.

The organic compost mixture (112) generally includes organic compost material, organic matter, or both. It may also include other components, such as water, liquids, and/or additives. Organic compost material is any product of microbial composting or microbial metabolism of organic matter (both generally referred to herein as “composting”). Such composting occurs when organic matter decays and decomposes, whether naturally or assisted with chemical or microbial additives, into organic compost material. Thus, the organic matter is a precursor to the organic compost material.

Any organic substance may be a suitable source of organic matter to generate the organic compost material. Examples of suitable organic matter for composting include, but are not limited to, human biosludge, human waste, animal waste, animal carcasses, tires, food, cellulosic materials, lignin, construction and demolition materials, plant matter, wood chips, straw, peat, cardboard, paper, coffee grounds, coir, cocoa shell, garden waste, leaves, grass, seaweed, manure, mushrooms, tree bark, eggshells, and the like. In one aspect of the novel system and method, the organic matter contains up to about 90% cellulose, such as grass, algae, cotton, wood pulp, wood chips, paper, cardboard, straw, and the like. One of the benefits of using cellulosic organic matter as a source material for production of humic substances instead of lignite is that the cellulose increases the quantity and production time of humic substances, and is a precursor to and preliminary component of fulvic acid.

The organic matter used in the novel system and process may produce organic compost material by either aerobic or anaerobic composting of the organic matter. Aerobically generated organic compost material is especially beneficial in the production and extraction of fulvic acid. One of the byproducts of aerobic composting is carbon dioxide, which is trapped in the organic compost material and therefore can become a part of the extracted liquid effluent (113). Anaerobic composting typically produces nitrogen and ammonia as byproducts, but the ammonia can be easily converted into ammonium nitrate, a common component of fertilizers, by those of skill in the art. Thus, the resulting liquid effluent (113) can contain not only humic substances, such as fulvic acid and humic acid, but also ammonium nitrate.

In another embodiment of the novel system and method, the organic compost mixture (112) comprises a compost windrow. A windrow is a long heap or pile of organic matter and/or organic compost material, often in a substantially triangular or mounded shape, for composting of the organic matter into organic compost material. While windrows may be of any shape or size, they are often hundreds of feet long and several feet tall. The size, shape, and contents of the windrow can be selected by those of skill according to the desired composting process parameters.

The liquid (111) combined with the organic compost mixture (112) can be any type of liquid in which fulvic acid can dissolve. In one aspect, the liquid (111) comprises water, which dissolves fulvic acid and also provides moisture to the organic compost mixture (112) necessary for any microbes in the organic compost mixture (112) to carry out the composting process. However, the liquid (111) may be any liquid or solution capable of dissolving fulvic acid. In one aspect, the liquid (111) combined with the organic compost mixture (112) is ionic water, which also aids in stabilizing and killing harmful pathogens in the organic compost mixture (112). The liquid (111) combined with the organic compost mixture (112) may also contain other useful and beneficial components, whether for treatment of harmful pathogens, to aid in the composting process, or as additives as may be desired in the final effluent product.

The liquid (111) can be added to the organic compost mixture (112) by various methods. In one embodiment, the liquid (111) is sprayed or applied to the surface of the organic compost mixture (112). This method is often used when the organic compost mixture (112) is a windrow. In another embodiment, the liquid (111) is added to the organic compost mixture (112) by mixing it with the organic compost mixture (112) in a mixer or other apparatus configured for mixing solids and liquids.

The liquid (111) can be added to the organic compost mixture (112) all at once, or at different times and intervals. The composting process usually requires some moisture content, so as the composting progresses the liquid (111) may need to be added periodically to ensure that the organic compost mixture (112) has the necessary moisture content. In another embodiment, the liquid (111) is added to the organic compost mixture (112) in a mixer or other conduit that mixes the two components.

The quantity of liquid (111) added to the organic compost mixture (112) can vary, and can be determined based on a number of different factors. In one aspect, the liquid (111) added to the organic compost mixture (112) will be determined by the composting process requirements. The amount of liquid (111) added can also vary depending on the moisture content found in the organic compost mixture (112). In one aspect, where water is used as the liquid (111), the ratio of water to organic compost material (112) is approximately one-to-one (1:1) by weight.

In another embodiment, the quantity of liquid (111) added is the amount necessary to saturate the organic compost mixture (112). In yet another embodiment, the amount of liquid (111) added to the organic compost mixture exceeds the saturation level of the organic compost mixture (112), thus resulting in excess or waste liquid runoff. The amount of liquid (111) to be added can vary depending on the desired amount of excess or waste runoff, as well as on the desired concentration of humic substances, including fulvic acid, in the resulting liquid effluent (113).

The liquid component (not shown) of the organic compost mixture (112) can be extracted in a number of different methods. In one embodiment, the liquid component is extracted by collecting the liquid component percolating through the organic compost mixture (112). The liquid component may percolate naturally through the organic compost mixture (112), such as by gravity. In another embodiment, percolation may be induced, such as be adjusting ambient pressure, temperature, or humidity. In another embodiment, percolation may be induced by adding liquid (111) to the organic compost mixture (112) in an amount that exceeds the saturation level of the organic compost mixture (112). When the organic compost mixture (112) is saturated, the liquid (111) added in excess of the saturation level cause excess liquid in the organic compost mixture (112) to percolate through and from the organic compost mixture (112). The percolating liquid effluent (113) is then collected by any means known to those of skill in the art, such as by allowing the liquid effluent (113) to flow or drip into or through a defined channel, collecting in a receiving tank, or by pumping. Indeed, any process or technique known to those of skill in the art can be employed to collect or gather effluent (113) from the organic compost mixture (112).

In another embodiment, shown in FIG. 2, the liquid component of the organic compost mixture (212) is collected from a slurry (213) created by adding liquid (211) to organic compost mixture (212) according to the methods previously described. The slurry (213) is also an organic compost mixture. The liquid component is separated from the solid components by means of a separator (215). Suitable separators (215) generally include any type of apparatus capable of separating solids from liquids. Examples of a suitable separator (215) include, but are not limited to, a centrifuge, belt press, filter press, membrane press, or the like, or any combination of them. Once the slurry (213) is added into the separator (215), the separator (215) separates the solid components from the liquid component. The separated liquid component thus becomes the liquid effluent (216), which contains humic substances, including fulvic acid and humic acid.

In one embodiment, the separator (215) comprises a centrifuge. Typically, a stationary or continuous centrifuge will provide suitable separation of the liquid component from the solid components. Continuous centrifuges allow the continuous addition of slurry (213), the continuous removal of the liquid component, and the discontinuous, semicontinuous or continuous removal of the solid components. These types of centrifuges include, but are not limited to, tubular bowl centrifuges, continuous scroll centrifuges, and continuous multichamber disk-stack centrifuges. Semi-continuous centrifuges may also be used. Indeed, any type of centrifuge that allows the separation of solids from liquids may achieve the desired results. Other possible centrifuges include basket centrifuges, disk centrifuges, high speed centrifuges, industrial centrifuges, laboratory centrifuges, and ultracentrifuges.

In another embodiment of the novel method, the separator (215) comprises a belt press. A belt press is generally a dewatering device utilizing two opposing synthetic fabric belts, revolving over a series of rollers to squeeze liquid from the slurry (213). The belt press dewaters the slurry (213) by applying an increasing surface pressure to the slurry (213) as it passes between moving belts and/or a series of press rollers. While most belt press processes are intended to capture the solids while merely reusing or disposing of the waste liquid, in the novel process the liquid component drawn off from the slurry (213) by the belt press is captured as the desired effluent product (216). Any type of belt press that separates liquids from solids is suitable for the novel process.

In another embodiment, the separator (215) comprises a filter press. A filter press is beneficial for use with the novel method because it is a highly efficient, compact, dewatering device for separating solids from liquid slurries. In yet another embodiment, the separator (215) comprises a membrane press. Any type of filter press or membrane press that separates liquids from solids is suitable for the novel process. Indeed, any process or apparatus known to those of skill in the art for separating liquids from solids may be used in the novel process and system.

Regardless of the type of separator (215) used, the resulting liquid effluent (216) contains humic substances. Fulvic acid generally is the most abundant component of the liquid effluent (216). Other components of the effluent (216) include minerals, humates, fulvates, and salts formed during the organic composting process or the novel process described herein. Humates are mineral salts formed with humic acid, and fulvates are mineral salts formed with fulvic acid. Thus, in addition to fulvic acid and humic acid, the resulting effluent contains many minerals and nutrients beneficial to plant growth and health. As mentioned previously, the resulting effluent (216) may also contain ammonium nitrate and other byproducts of the composting process.

The novel system and process described herein may also be modified in many different aspects to produce the desired product. For example, in one embodiment of the novel system and method, shown in FIG. 3, the slurry (313) may optionally pass through a strainer or filter (314) to remove the larger particulate solids prior to entrance of the slurry (313) into the separator (315). This enhances the ability of the separator (315) to separate the solid components from the liquid component by removing the larger solid components prior to passing through the separator (315). Any type of strainer can be employed to effect this filtering process.

In another embodiment, also shown in FIG. 3, the concentration of fulvic acid in the resulting effluent (316) can be optimized by reusing the effluent (316) in the system and process. In this embodiment, after the slurry (313) has passed through the separator (315) and the liquid component separated from the solid components, the effluent (316) drawn off the separator (315) is re-mixed with organic compost mixture (312) or slurry (313) for separation of the solid components from the liquid component in the organic compost mixture (312) or slurry (313) by means of the separator (315). The organic compost mixture (312) that is re-mixed with the effluent may be new or additional organic compost mixture, or may be the original organic compost mixture drawn off from the separator. In one embodiment, the effluent (316) is added to the organic compost mixture (312) to achieve approximately a 3:1 ratio by weight of effluent (316) to solid components prior to the second separation step. This ratio may be adjusted as necessary to achieve optimum results. In one embodiment, this second separation step can be carried out on a second separator. The additional separation step may also be carried out on any number of sequential separators until the desired concentration and composition of the resulting effluent (316) is achieved. By repeating the separation step in the process and reusing the effluent (316), the resulting concentration of fulvic acid in the effluent (316) can be doubled or increased many times more than would result with only one pass through a separator (315).

In another embodiment, also shown in FIG. 3, the solid components (317) separated from the liquid component by the separator (315) may also be used or reused in various applications. In one embodiment, the resulting solids (317) are again combined with liquid (311) to create a slurry (313) that is then run through a separator (315) to separate out the humic substances, including fulvic acid, that remained in the solids and did not separate with the liquid effluent (316) during the prior separation. The same procedures as described above for reuse of the effluent (316) can be employed on the separated solid components (317). Indeed, this process may be repeated on the solid components (317) multiple times in order to achieve a maximum or desired extraction of the humic substances, including fulvic acid.

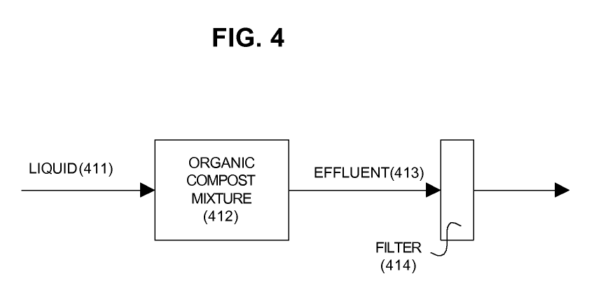

In another aspect of the novel system and method, once the effluent containing humic substances, including fulvic acid, has been collected from the organic compost mixture, it can then be prepared for use. For example, in one embodiment shown in FIG. 4, the effluent (413) is filtered or strained by a filter (414) prior to use to remove any remaining large solid components. In one embodiment, the filter (414) comprises a 50 micron filter. However, any size and number of filters (414) may be employed, depending on the desired level of filtration of the effluent (413).

In another embodiment, not shown in the figures, the system and process optionally includes a treatment step to kill pathogens in the effluent and stabilize the effluent for use. The organic compost mixture, slurry, and/or effluent may contain any number of harmful pathogens, particularly where the organic matter used includes manure and other blackwaste. The treatment carried out on the effluent may occur at any stage of the process, including prior to or after separation of the liquid and solid components, and prior to or after filtration of the effluent. Any process known to those of skill in the art can be used for treatment of the effluent. In one embodiment, copper sulfate is added to the effluent as a treatment to kill pathogens and stabilize the effluent. In another embodiment, the effluent is treated by adding microbes selected for their capacity to kill harmful pathogens. In another embodiment, the treatment step comprises one or more heat processes to kill pathogens, including, but not limited to, pasteurization or thermophilic composting. These heat processes may occur during the composting process, or they may occur after collection of the effluent from the organic compost mixture, or both.

While the effluent resulting from any of the processes described herein may be the final product and ready for use, other optional processes may be carried out to prepare the resulting product for specific uses. For example, the effluent may be dried to create a dry powder. Any process known to those of skill in the art can be employed to effectuate this drying process. Other components and additives may also be added to the product, depending on the desired composition and use of the product. Examples include, but are not limited to, fertilizer components, urea, and potassium. Such additives, in combination with the fulvic acid and humic acid, provide valuable benefits and advantages for plant growth and nutrition.

The effluent from the above-described systems and methods results in a novel product that contains a high concentration of humic substances, particularly fulvic acid, and beneficial plant nutrients. Generally, the composition of the final product includes fulvic acid, which in one embodiment comprises at least 4% of the total product by weight, and in one aspect comprises approximately 4% to 10%, and in another aspect comprises at least 7%, and in another aspect comprises approximately 7% to 10%. The product also comprises humic acid up to approximately 3% of the total product by weight, and in another aspect comprises humic acid at approximately 0.5% to approximately 2.5% by weight of the total product.

The novel product also contains a large amount of necessary plant nutrients, including both macronutrients and micronutrients. For example, the novel product contains appreciable quantities of phosphorous, potassium, calcium, magnesium, sulfur, boron, copper, iron, chlorine, manganese, molybdenum, and zinc. The product also has little to no heavy metals because the product is not produced from lignite or mineral ores. Standard methods for production of humic substances, including fulvic acid, from lignite, coal, and mineral sources result in a product that contains higher levels of heavy metals, such as lead and cadmium, because the acids used in these processes do not strip the humic substances of heavy metals. However, the novel fulvic acid product described herein contains very little heavy metals because the source material for the product contains very few heavy metals. For example, the product generally contains less than approximately 0.1 ppm of cadmium, and specifically less than approximately 0.061 ppm, and more specifically less than approximately 0.020 ppm. The product also contains less than approximately 0.1 ppm of lead, and specifically less than 0.060 ppm, and more specifically less than 0.055 ppm.

The systems, methods, and products described herein can be better understood with a description of the following examples. It should be noted, however, that the following examples are to serve only as examples and should in no way provide limitations to the systems, methods, and products described herein.

### Example 1

An exemplary fulvic acid solution was prepared as follows. Water was combined with an organic compost mixture in the form and formulation of compost windrows formulated for mushroom growth. The compost windrows contained rye straw (85-90% by weight), chicken manure, peat, gypsum, and shaft from alfalfa seeds. Water was added to the exterior surface of compost windrows in amounts that exceeded the saturation level of the compost windrows. The excess water effluent that escaped out of the organic compost mixture windrows was collected in defined channels at the bases of the windrows. This water effluent was then passed through a 50 micron filter, and then treated to kill harmful pathogens by adding copper sulfate to the effluent. The resulting concentration of fulvic acid and humic acid, micronutrients, and macronutrients in the product was as shown in Table 1 below. The concentration of fulvic acid and humic acid were measured by spectrophotometric analysis.

TABLE 1

| Component | Concentration (ppm) |
|---|---|
| Fulvic Acid* | 9.25% |
| Humic Acid* | 0.77% |
| Phosphorous | 89.70 |
| Potassium | 7,290.00 |
| Calcium | 274.00 |
| Magnesium | 129.00 |
| Sulfur | 739.00 |
| Boron | 1.54 |
| Copper | 0.46 |
| Iron | 5.66 |
| Chlorine | 428.00 |
| Manganese | 0.76 |
| Molybdenum | 0.21 |
| Zinc | 1.89 |

*Concentration measured as % by weight

### Example 2

An exemplary fulvic acid solution was prepared as follows. Water was combined with an organic compost mixture in the form and formulation of organic compost material designed and used as a bed for mushroom growth. The organic compost material was generated from organic matter comprising rye straw (85-90% by weight), chicken manure, peat, gypsum, and shaft from alfalfa seeds. The organic compost material was used approximately 1-day after mushrooms growing on the bed were harvested. Water was mixed with the organic compost material to create a slurry. The slurry then passed through a centrifuge separator to separate the slurry's solid components from its liquid component. The resulting concentration of fulvic acid and humic acid in the liquid product was as shown in Table 2 below. The concentration of fulvic acid and humic acid were measured by spectrophotometric analysis.

TABLE 2

| | Concentration |
|---|---|
| Component | (% by weight) |
| Fulvic Acid | 7.19% |
| Humic Acid | 2.28% |

### Example 3

An exemplary fulvic acid solution was prepared as follows. Water was combined with an organic compost mixture in the form and formulation of organic compost material designed and used as a bed for mushroom growth. The organic compost material was generated from organic compost mixture containing rye straw (85-90% by weight), chicken manure, peat, gypsum, and shaft from alfalfa seeds. The organic compost material was used approximately 14-days after mushrooms growing on the bed were harvested. Water was mixed with the organic compost material to create a slurry. The slurry then passed through a centrifuge separator to separate the slurry's solid components from its liquid component. The resulting concentration of fulvic acid and humic acid in the liquid product was as shown in Table 3 below. The concentration of fulvic acid and humic acid were measured by spectrophotometric analysis.

TABLE 3

| | Concentration |
|---|---|
| Component | (% by weight) |
| Fulvic Acid | 8.71% |
| Humic Acid | 0.92% |

### Example 4

An exemplary fulvic acid solution was prepared as follows. Water was combined with an organic compost mixture in the form and formulation of organic compost material designed and used as a bed for mushroom growth. The organic compost material was generated from organic compost mixture containing rye straw (85-90% by weight), chicken manure, peat, gypsum, and shaft from alfalfa seeds. The organic compost material was used approximately 10-weeks after mushrooms growing on the bed were harvested. Water was mixed with the organic compost material to create a slurry. The slurry then passed through a belt press separator to separate the slurry's solid components from its liquid component. The resulting composition of the product was as shown in Table 4 below. The concentration of fulvic acid and humic acid were measured by spectrophotometric analysis.

TABLE 4

| Component | Concentration (ppm) |
|---|---|
| Fulvic Acid* | 9.06% |
| Humic Acid* | 0.51% |
| Phosphorous | 60.80 |
| Potassium | 18,900.00 |
| Calcium | 1,690.00 |
| Magnesium | 407.00 |
| Sulfur | 4,720.00 |
| Boron | 1.03 |
| Copper | 0.12 |
| Iron | 5.28 |
| Manganese | 1.18 |
| Molybdenum | 0.15 |
| Zinc | 0.39 |

*Measured as % by weight

### Example 5

An exemplary fulvic acid solution was prepared as follows. Water was combined with an organic compost mixture in the form and formulation of organic compost material designed and used as a bed for mushroom growth. The organic compost material was generated from organic compost mixture containing rye straw (85-90% by weight), chicken manure, peat, gypsum, and shaft from alfalfa seeds. Water was mixed with the organic compost material to create a slurry. The slurry then passed through a centrifuge separator to separate the slurry's solid components from its liquid component. The resulting concentration of fulvic acid in the liquid product was approximately 4% by weight. This liquid product was then reused by combining it with another similar organic compost mixture, which was then run through the centrifuge. The concentration of fulvic acid in the liquid product after the second separation in the centrifuge was approximately 7.6% by weight.

The product produced according to the systems and methods described herein can be used for many different purposes, including agriculture, farming, gardening, and horticulture. Examples of these uses include, but are not limited to, lawns, flower and vegetable gardens, trees, vines, ornamentals, landscaping, parks, golf greens, parks, and newly laid top soils and/or turf, sports fields, fruit trees, and the like. In these applications, the product stimulates plant growth, revitalizes distressed plants, increases mineral and nutrient uptake, and improves roots.

The product can also be used as a pesticide to repel pests and insects. The inventors have observed that distressed plants and plants in poor nutrition attract pests and insects, which further damage the plants and consume much needed nutrients. For example, bark beetles often attack trees that are already weakened by disease, drought, smog, other beetles, or physical damage. The inventors have discovered that by applying the product to the plant environment of distressed plants, the plants become healthy again, thereby repelling pests and insects. Healthy trees may put up defenses by producing resin or latex, which may contain a number of insecticidal and fungicidal compounds that can kill or injure attacking insects, or simply immobilize and suffocate them with the sticky fluid. These results can be achieved with any type of distressed plant to repel almost any type of insect. In one particular embodiment, the product can be applied to pine trees and other coniferous trees to eradicate the bark beetle and other insects and pests. It In another embodiment the product is used as an additive in other beneficial chemicals, substances, and compounds, including, but not limited to, fertilizers, soil amendments, herbicides, nutrients, pesticides, insecticides, fungicides, and defoliants. In another embodiment, the product is used as an additive in microbial mixtures used for composting.

In one embodiment, the product is used by applying it to a plant environment, which includes a plant and all of its parts, such as roots, stems, leaves, and fruit, the soil and air from which the plant draws water and nutrients. The product may be applied to the plant environment in many different forms. For example, in one embodiment the product as produced by the foregoing novel systems and methods is applied to the plant environment directly without modification. In another embodiment, the product is applied to the plant environment as an amendment with fertilizer. The product may be applied to the plant environment either in dry form, such as in a powder or bricks, or in liquid solution form. In another embodiment, the product is applied to the plant environment as a component of another beneficial chemical, substance, or compound, as mentioned above.

The product may be applied to the plant environment by many different means, including, but not limited to, spraying, irrigation, fertigation, flood irrigation, drip irrigation, sprinkler irrigation, and the like. In one embodiment, the product is applied directly to plants as a foliar spray. In another embodiment, the product may be applied to the plant environment in solid form by spreading, burying the product in the soil, or placing the product in or on the soil.

The above-described novel systems and methods have several benefits and advantages over current systems and methods for separating and producing humic substances and fulvic acid. Organic compost material and organic matter, unlike the traditional sources for humic substances and fulvic acid (i.e. coal, lignite, and other mineral ores), is not finite. Rather, organic compost material and organic matter are essentially renewable resources, based on the carbon life cycle. There is little risk of depleting these sources of humic substances.

Additionally, the novel process is generally much faster than current technology for separating and producing humic substances, which require hard rock mining and separation of humic substances from lignite, coal, and hardrock mineral ores. Thus, the novel process also avoids the harmful effects of the mining process on the environment. It is also much cheaper to operate and produce fulvic acid and humic substances from organic matter and organic compost material than from tradition hardrock sources. Another advantage of this process is that it can handle a much wider range of materials, virtually working with any type of organic matter or organic compost material. This process also does not require the use or generation of new decomposition microbes or microorganisms to produce the necessary organic compost material. Rather, the process relies on the use of existing methods and technologies for generating suitable organic compost material. The novel process described herein does not require the use of acids or bases to leach out the humic substances. Additionally, this process allows for commercial and large-scale production because organic matter and organic compost material are available in large, commercial quantities for use in the novel systems and processes described herein. Finally, the novel systems and processes virtually eliminate all heavy metals from the final product. Existing extraction technology is not able to satisfactorily remove all heavy metals from the humic substances. Thus, the final product contains a beneficial mixture of humic substances, particularly fulvic acid, without heavy metals or the need to remove heavy metals.

The preceding description has been presented only to illustrate and describe exemplary embodiments of the present system and methods of producing humic substances and fulvic acid, and the resulting fulvic acid product and its uses. It is not intended to be exhaustive or to limit the system, methods, and products to any precise form or embodiment disclosed. Many modifications and variations are possible in light of the above teaching. It is intended that the scope of the products and processes be defined by the following claims.

What is claimed is:

1. A method of producing fulvic acid and humic acid, comprising extracting liquid from an organic compost mixture.

2. The method of claim 1, further comprising adding a source liquid to said organic compost mixture prior to said extracting.

3. The method of claim 2, wherein said source liquid comprises water.

4. The method of claim 1, wherein said extracting comprises collecting liquid percolating from said organic compost mixture.

5. The method of claim 4, further comprising inducing said liquid to percolate from said organic compost mixture.

6. The method of claim 5, wherein said inducing comprises adding source liquid to said organic compost mixture in excess of the saturation level of said organic compost mixture.

7. The method of claim 2, wherein said extracting comprises separating said liquid from solids in the organic compost mixture.

8. The method of claim 7, wherein said separating comprises passing said organic compost mixture through a separator configured to separate liquids and solids.

9. The method of claim 8, wherein said separator comprises a centrifuge, belt press, filter press, membrane press, or a combination thereof.

10. The method of claim 1, wherein said organic compost mixture comprises cellulosic organic matter.

11. The method of claim 1, further comprising treating said liquid extracted from the organic compost mixture.

12. The method of claim 1, further comprising filtering said liquid extracted from the organic compost mixture.

13. A system for producing fulvic acid and humic acid, comprising an organic compost mixture.

14. The system of claim 13, further comprising a source liquid configured to dissolve fulvic acid.

15. The system of claim 14, wherein said source liquid is water.

16. The system of claim 13, further comprising a separator.

17. The system of claim 16, wherein said separator comprises a centrifuge, belt press, filter press, membrane press, or a combination thereof.

18. A composition, comprising:
fulvic acid, comprising at least approximately 4% of the composition by weight; and
humic acid, comprising less than approximately 3% of the composition by weight.

19. The composition of claim 18, wherein fulvic acid comprises approximately 4% to approximately 10% of the composition by weight.

20. The composition of claim 18, wherein fulvic acid comprises at least approximately 7% of the composition by weight.

21. The composition of claim 20, wherein fulvic acid comprises approximately 7% to approximately 10% of the composition by weight.

22. The composition of claim 18, further comprising plant nutrients.

23. A method for eradicating a pest from a plant, comprising applying to a plant environment a composition comprising at least approximately 4% fulvic acid by weight.

24. The method of claim 23, wherein said pest comprises a bark beetle and said plant is coniferous.

25. The method of claim 23, wherein said composition comprises at least approximately 7% fulvic acid by weight.

26. A method for improving plant health, comprising applying to a plant environment a composition comprising at least approximately 4% fulvic acid by weight.
